(19) United States
(12) Patent Application Publication
(10) Pub. No.: US 2007/0142803 A1
(43) Pub. Date: Jun. 21, 2007

(54) Articles comprising superabsorbent polymer compositions

(76) Inventors: Dave Allen Soerens, Neenah, WI (US); Timothy James Van Himbergen, Appleton, WI (US); Michael David Meisner, DePere, WI (US)

Correspondence Address: KIMBERLY-CLARK WORLDWIDE, INC., 401 NORTH LAKE STREET, NEENAH, WI, 54956, US

(21) Appl. No.: 11/305,592

(22) Filed: Dec. 15, 2005

(51) Int. Cl. A61F 13/15

(52) U.S. Cl. 604/368

(57) ABSTRACT

An absorbent article comprises a backsheet and an absorbent layer, where the absorbent layer comprises a support substrate and a superabsorbent polymer composition positioned adjacent to the substrate. The absorbent layer may also include an additional substrate, and the article may additionally include a topsheet. The superabsorbent polymer composition comprises a water-soluble ionic polymer capable of sufficient non-radiative crosslinking within about 10 minutes at a temperature of about 120° C. or less to reach an absorbent capacity of at least 1 g/g as measured by the Centrifuge Retention Capacity Test. In addition, the absorbent layer is restretched. The result is an absorbent article which exhibits improved performance as well as greater comfort and confidence among the user.

### BACKGROUND

Articles are an essential part of people's lives. For example, absorbent articles can be useful for absorbing many types of fluids, including fluids secreted or eliminated by the human body. Superabsorbent materials are frequently used in absorbent articles to help improve the absorbent properties of such articles. Superabsorbent materials are generally polymer based and are available in many forms, such as powders, granules, microparticles, films and fibers, for example. Upon contact with fluids, such superabsorbent materials swell by absorbing the fluids into their structures. In general, superabsorbent materials can quickly absorb fluids insulted into such articles, and can retain such fluids to prevent leakage and help provide a dry feel, even after fluid insult.

There is continuing effort to improve the performance of articles, such as absorbent articles. One desire is to make absorbent articles thinner. Another desire is to make absorbent articles more flexible. Still another desire is to increase the integrity of absorbent articles, in both a wet and dry condition. Yet another desire is to increase the absorbent intake and/or the absorbent capacity of such articles. One method for improving absorbent properties is to increase the amount of superabsorbent material in the article. However, an increase in superabsorbent material content can increase the thickness of the article, and likewise can decrease the flexibility and/or the integrity of the article. Therefore, there is a need for an article, such as an absorbent article, which achieves desired absorbent properties while maintaining desired thinness and/or flexibility and/or integrity in both a wet and dry condition.

The use of hydrophilic polymers may be desirable in absorbent articles. A known approach for making hydrophilic polymers more functional upon exposure to aqueous fluid is to crosslink the water-soluble polymers. As a result of crosslinking, the material becomes swellable, and no longer soluble, in aqueous fluid. However, crosslinked polymers are difficult to apply to substrates or to establish intimate contact with surfaces because the crosslinked polymers are solid materials and have little or no ability to flow. In addition, some of the crosslinked materials are fairly stiff, and inhibit the flexibility of the absorbent product.

What is therefore needed is a hydrophilic polymer that has latent crosslinking capability and which can be produced at an attractive cost. Such polymers could be easily applied, like a water-soluble polymer, since the hydrophilic polymer would be capable of flow prior to crosslinking. Latent crosslinking capability would also provide a simple means of crosslinking the polymer after the polymer has established intimate contact with substrates or has formed a desired final shape or form. There is also a need or desire for an absorbent article comprising such a polymer that has a suitable level of flexibility.

### SUMMARY

In response to the needs discussed above, an absorbent article, such as a disposable absorbent article, such as a sanitary napkin, a wound-care article or furniture pad is provided. More particularly, the article can comprise a backsheet and optionally a topsheet, and an absorbent layer that includes a superabsorbent polymer composition and a support substrate. The absorbent layer can be in contact with a planar surface of the backsheet and/or the optional topsheet. Alternatively, the superabsorbent polymer composition may be positioned between both a backsheet and a topsheet. In other aspects, the absorbent layer may function as the backsheet or as the topsheet. In still other aspects, the absorbent article of the present invention can be selected from personal care absorbent articles, health/medical absorbent articles or household/industrial absorbent articles.

Provided for by this invention is an absorbent article comprising a backsheet and an absorbent layer adjacent to and in facing relationship with the backsheet. The absorbent layer includes a support substrate and a superabsorbent polymer composition formed from a water-soluble ionic polymer capable of sufficient non-radiative crosslinking within about 10 minutes at a temperature of about 120° C. or less to reach an absorbent capacity of at least 1 g/g as measured by the Centrifuge Retention Capacity Test. In addition, the absorbent layer has been restretched. In one particular aspect, the superabsorbent polymer composition comprises about 15 to about 99.9% by mass monoethylenically unsaturated polymer units, about 0.1 to about 20% by mass ester units selected from the group consisting of acrylate and methacrylate ester units that include an alkoxysilane functionality, and zero to about 75% by mass of units selected from the group consisting of polyolefin glycol and polyolefin oxide units.

In particular features, the superabsorbent polymer composition has been substantially fissured. In some aspects, the absorbent layer has a density of about 0.1 g/cc or less. In other aspects, the absorbent layer exhibits a decrease in stiffness as measured by the Plate Stiffness Test of at least about 50% when compared to a similar unrestretched absorbent layer. In particular aspects, the absorbent layer has a stiffness of about 2 N·mm or less as measured by the Plate Stiffness Test. In yet other aspects, the absorbent layer exhibits an increase in absorbent capacity as measured by the Composite Centrifuge Retention Capacity Test of at least about 30% when compared to a similar unrestretched absorbent layer. In particular aspects, the absorbent layer has an absorbent capacity of at least about 800 g/m² as measured by the Composite Centrifuge Retention Capacity Test. In other particular aspects, the absorbent layer has a thickness of about 1 mm or less. In yet other particular aspects, the absorbent layer has a CD wet strength of at least about 11 N as measured by ASTM D-5034.

In some aspects, the absorbent article optionally includes a topsheet, where the topsheet is positioned such that the absorbent layer is positioned between the topsheet and the backsheet. In other aspects, at least one of the backsheet, the absorbent layer and the topsheet is stretchable.

In some aspects, the absorbent layer further comprises an additional substrate which may be positioned such that the superabsorbent polymer composition is positioned between the support substrate and the additional substrate. In one aspect of this embodiment, the absorbent layer has a density of about 0.3 g/cc or less. In other aspects of this embodiment, the absorbent layer exhibits a decrease in stiffness as measured by the Plate Stiffness Test of at least about 50% when compared to a similar unrestretched absorbent layer. In particular aspects of this embodiment, the absorbent layer has a stiffness of about 2 N·mm or less as measured by the Plate Stiffness Test. In another aspect of this embodiment, the absorbent layer exhibits an increase in absorbent capacity index as measured by the Composite Centrifuge Retention Capacity Index Test of at least about 10% when compared to a similar unrestretched absorbent layer. In particular aspects of this embodiment, the absorbent layer has an absorbent capacity index of at least about 7 as measured by the Composite Centrifuge Retention Capacity Index Test. In other particular aspects of this embodiment, the absorbent layer has a thickness of about 2 mm or less. In yet other particular aspects of this embodiment, the absorbent layer has a CD wet strength of at least about 11 N as measured by ASTM D-5034.

The result is an article which exhibits improved performance as well as greater comfort and confidence among the user.

Numerous other features and advantages of the present invention will appear from the following description. In the description, reference is made to exemplary embodiments of the invention. Such embodiments do not represent the full scope of the invention. Reference should therefore be made to the claims herein for interpreting the full scope of the invention.

### FIGURES

The foregoing and other features, aspects and advantages of the present invention will become better understood with regard to the following description, appended claims and accompanying drawings where:

FIGS 1A and 1B are each a cross-section of an absorbent layer of the present invention having a substrate layer and an absorbent layer.

FIG. 1C is a cross-section of an absorbent layer of the present invention having a substrate layer impregnated with an absorbent material.

FIGS. 2A and 2B are each a cross-section of an absorbent layer of the present invention having a substrate layer impregnated with the absorbent material and an additional layer.

FIGS. 2C and 2D are each a cross-section of an absorbent layer of the present invention having a substrate layer, an absorbent layer and an additional layer.

FIG. 3 is an apparatus for making an absorbent layer of the present invention.

FIG. 4 is an apparatus for restretching an absorbent layer of the present invention.

FIG. 5 is a representative, partially cut-away, top view of a body-facing side of an absorbent article which includes the superabsorbent polymer composition of the present invention.

FIG. 6A is a cross-section side view of an absorbent bandage of the present invention.

FIG. 6B is a top perspective view of an absorbent bandage of the present invention.

FIG. 7 is a top perspective view of an absorbent bed or furniture liner of the present invention.

Repeated use of reference characters in the present specification and drawings is intended to represent the same or analogous features or elements of the present invention.

### DEFINITIONS

It should be noted that, when employed in the present disclosure, the terms “comprises,” “comprising” and other derivatives from the root term “comprise” are intended to be open-ended terms that specify the presence of any stated features, elements, integers, steps, or components, and are not intended to preclude the presence or addition of one or more other features, elements, integers, steps, components, or groups thereof.

The term “absorbent article” generally refers to a device which can absorb and contain fluids. For example, personal care absorbent articles refer to devices which are placed against or near the skin to absorb and contain the various fluids discharged from the body. The term “disposable” is used herein to describe absorbent articles that are not intended to be laundered or otherwise restored or reused as an absorbent article after a single use. Examples of such disposable absorbent articles include, but are not limited to, personal care absorbent articles, health/medical absorbent articles, and household/industrial absorbent articles.

The term “coform” is intended to describe a blend of meltblown fibers and cellulose fibers that is formed by air forming a meltblown polymer material while simultaneously blowing air-suspended cellulose fibers into the stream of meltblown fibers. The coform material may also include other materials, such as superabsorbent materials. The meltblown fibers containing wood fibers are collected on a forming surface, such as provided by a foraminous belt. The forming surface may include a gas-pervious material, such as spunbonded fabric material, that has been placed onto the forming surface.

The phrase “complex fluids” describes fluids generally characterized as being viscoelastic comprising multiple components having inhomogeneous physical and/or chemical properties. It is the inhomogeneous properties of the multiple components that challenge the efficacy of an absorbent or adsorbent material in the handling of complex liquids. In contrast with complex fluids, simple fluids, such as, for example, urine, physiological saline, water and the like, are generally characterized as being relatively low-viscosity and comprising one or more components having homogeneous physical and/or chemical properties. As a result of having homogeneous properties, the one or more components of simple fluids behave substantially similarly during absorption or adsorption, although some components of the simple fluids may be absorbed or adsorbed more readily than others. Although a complex liquid is generally characterized herein as including specific components having inhomogeneous properties, each specific component of a complex liquid generally has homogeneous properties. Consider for example a representative complex body-liquid having three specific components: red blood cells, blood protein molecules and water molecules. Upon examination, one skilled in the art could easily distinguish between each of the three specific components according to their generally inhomogeneous properties. Moreover, when examining a particular specific component such as the red blood cell component, one skilled in the art could easily recognize the generally homogeneous properties of the red blood cells

The term “dry” when used in the phrase “dry superabsorbent polymer composition” or “dry absorbent layer”, or similar phrases refers to a crosslinked superabsorbent polymer composition layer that has a solids content of about 92% by weight or greater.

The terms “elastic,” “elastomeric” and “elastically extensible” are used interchangeably to refer to a material or composite that generally exhibits properties which approximate the properties of natural rubber. The elastomeric material is generally capable of being extended or otherwise deformed, and then recovering a significant portion of its shape after the extension or deforming force is removed.

The term “extensible” refers to a material that is generally capable of being extended or otherwise deformed, but which does not recover a significant portion of its shape after the extension or deforming force is removed.

The term “fluid impermeable,” when used to describe a layer or laminate, means that fluid such as water or bodily fluids will not pass substantially through the layer or laminate under ordinary use conditions in a direction generally perpendicular to the plane of the layer or laminate at the point of fluid contact.

The term “health/medical articles” includes a variety of professional and consumer health-care products including, but not limited to, products for applying hot or cold therapy, medical gowns (i.e., protective and/or surgical gowns), surgical drapes, caps, gloves, face masks, bandages, wound dressings, wipes, covers, containers, filters, disposable garments and bed pads, medical absorbent garments, underpads, and the like.

The term “household/industrial articles” include construction and packaging supplies, products for cleaning and disinfecting, wipes, covers, filters, towels, disposable cutting sheets, bath tissue, facial tissue, nonwoven roll goods, home-comfort products including pillows, pads, mats, cushions, furniture liners and pads, masks and body care products such as products used to cleanse or treat the skin, laboratory coats, cover-alls, trash bags, stain removers, topical compositions, pet care absorbent liners, laundry soil/ink absorbers, detergent agglomerators, lipophilic fluid separators, and the like.

The terms “hydrophilic” and “wettable” are used interchangeably to refer to a material having a contact angle of water in air of less than 90 degrees. The term “hydrophobic” refers to a material having a contact angle of water in air of at least 90 degrees. For the purposes of this application, contact angle measurements are determined as set forth in Robert J. Good and Robert J. Stromberg, Ed., in “Surface and Colloid Science—Experimental Methods,” Vol. II, (Plenum Press, 1979), herein incorporated by reference in a manner consistent with the present disclosure.

The term “insult target zone” refers to an area of an absorbent article or layer where it is particularly desirable for the majority of a fluid insult, such as urine, menses, or bowel movement, to initially contact. In particular, for an absorbent article or layer with one or more fluid insult points in use, the insult target zone refers to the area of the absorbent article or layer extending a distance equal to 15% of the total length of the composite from each insult point in both directions.

The term “knife over roll coating” refers to a process in which a knife is positioned, with a specified gap, above a substrate that is moving beneath the knife on a moving roll. In this manner, the knife spreads a specified thickness of coating material onto the substrate.

The term “meltblown fibers” refers to fibers formed by extruding a molten thermoplastic material through a plurality of fine, usually circular, die capillaries as molten threads or filaments into a high velocity, usually heated, gas (e.g., air) stream which attenuates the filaments of molten thermoplastic material to reduce their diameter. Thereafter, the meltblown fibers are carried by the high velocity gas stream and are deposited on a collecting surface to form a web of randomly disbursed meltblown fibers.

The terms “nonwoven” and “nonwoven web” refer to materials and webs of material having a structure of individual fibers or filaments which are interlaid, but not in an identifiable manner as in a knitted fabric. The terms “fiber” and “filament” are used herein interchangeably. Nonwoven fabrics or webs have been formed from many processes such as, for example, meltblowing processes, spunbonding processes, air laying processes, and bonded-carded-web processes. The basis weight of nonwoven fabrics is usually expressed in ounces of material per square yard (osy) or grams per square meter (gsm) and the fiber diameters are usually expressed in microns. (Note that to convert from osy to gsm, multiply osy by 33.91.)

The term “personal care articles” includes, but is not limited to, absorbent articles such as diapers, diaper pants, baby wipes, training pants, absorbent underpants, child care pants, swimwear, and other disposable garments; feminine care products including sanitary napkins, wipes, menstrual pads, menstrual pants, panty liners, panty shields, interlabials, tampons, and tampon applicators; adult-care products including wipes, pads such as breast pads, containers, incontinence products, and urinary shields; clothing components; bibs; athletic and recreation products; and the like.

The term “polymers” includes, but is not limited to, homopolymers, copolymers, such as for example, block, graft, random and alternating copolymers, terpolymers, etc. and blends and modifications thereof. Furthermore, unless otherwise specifically limited, the term “polymer” shall include all possible configurational isomers of the material. These configurations include, but are not limited to isotactic, syndiotactic and atactic symmetries.

The term “roll printing” or “roll coating” refers to a process in which the application of a deposited material, generally as a paste, onto a substrate is carried out by transferring the deposited material from a roll onto the substrate in a more or less uniform layer using one or more rolls, which may be engraved, and a pool cylinder. A doctor blade is used to scrape any excess deposited material from the rolls or substrate. The doctor blade may be flat or have a patterned edge such as slots or ridges.

The term “rotary screen printing” or “rotary screen coating” refers to a process that is a combination of roll printing or coating and screen printing or coating.

The term “screen printing” or “screen coating” refers to a method of applying a deposited material by forcing the material to be deposited through a screen that may have uniform openings or patterned openings.

The term “slot coating” refers to a process in which a slot die provides a thin, uniform coating on a substrate to be coated. In slot coating, the coating can be placed using an open gap in which the substrate to be coated is passed under the slot die, or a closed gap in which the slot die is aligned with a coating roll, such that there is a narrow gap or nip between the roller and slot die. The substrate to be coated is passed between the coating roll and the slot die.

The term “spontaneous” crosslinking refers to crosslinking which occurs without radiation, catalysis, or any other inducement other than the specified temperature of not more than about 150° C., such as not more than about 120° C., or not more than about 100° C.

The terms “spunbond” and “spunbonded fiber” refer to fibers which are formed by extruding filaments of molten thermoplastic material from a plurality of fine, usually circular, capillaries of a spinneret, and then rapidly reducing the diameter of the extruded filaments.

The term “stretchable” refers to materials which may be extensible or which may be elastically extensible.

These terms may be defined with additional language in the remaining portions of the specification.

### DETAILED DESCRIPTION

The present invention concerns an article, suitably an absorbent article, such as a sanitary napkin, a wound-care article or a furniture pad. More particularly, the article can comprise a backsheet and optionally a topsheet, and an absorbent layer that includes a superabsorbent polymer composition and a support substrate. The absorbent layer can be in contact with a planar surface of the backsheet and/or the optional topsheet. Alternatively, the superabsorbent polymer composition may be positioned between both a backsheet and a topsheet. In other aspects, the absorbent layer may function as the backsheet or as the topsheet. In still other aspects, the absorbent article of the present invention can be selected from personal care absorbent articles, health/medical absorbent articles or household/industrial absorbent articles.

Provided for by this invention is an absorbent article comprising a backsheet and an absorbent layer adjacent to and in facing relationship with the backsheet. The absorbent layer includes a support substrate and a superabsorbent polymer composition formed from a water-soluble ionic polymer capable of sufficient non-radiative crosslinking within about 10 minutes at a temperature of about 120° C. or less to reach an absorbent capacity of at least 1 g/g as measured by the Centrifuge Retention Capacity Test. In addition, the absorbent layer has been restretched. In one particular aspect, the superabsorbent polymer composition comprises about 15 to about 99.9% by mass monoethylenically unsaturated polymer units, about 0.1 to about 20% by mass ester units selected from the group consisting of acrylate and methacrylate ester units that include an alkoxysilane functionality, and zero to about 75% by mass of units selected from the group consisting of polyolefin glycol and polyolefin oxide units.

In particular features, the superabsorbent polymer composition has been substantially fissured. In some aspects, the absorbent layer has a density of about 0.1 g/cc or less. In other aspects, the absorbent layer exhibits a decrease in stiffness as measured by the Plate Stiffness Test of at least about 50% when compared to a similar unrestretched absorbent layer. In particular aspects, the absorbent layer has a stiffness of about 2 N·mm or less as measured by the Plate Stiffness Test. In yet other aspects, the absorbent layer exhibits an increase in absorbent capacity as measured by the Composite Centrifuge Retention Capacity Test of at least about 30% when compared to a similar unrestretched absorbent layer. In particular aspects, the absorbent layer has an absorbent capacity of at least about 800 g/m² as measured by the Composite Centrifuge Retention Capacity Test. In other particular aspects, the absorbent layer has a thickness of about 1 mm or less. In yet other particular aspects, the absorbent layer has a CD wet strength of at least about 11 N as measured by ASTM D-5034.

In some aspects, the absorbent article optionally includes a topsheet, where the topsheet is positioned such that the absorbent layer is positioned between the topsheet and the backsheet. In other aspects, at least one of the backsheet, the absorbent layer and the topsheet is stretchable.

In some aspects, the absorbent layer further comprises an additional substrate which may be positioned such that the superabsorbent polymer composition is positioned between the support substrate and the additional substrate. In one aspect of this embodiment, the absorbent layer has a density of about 0.3 g/cc or less. In other aspects of this embodiment, the absorbent layer exhibits a decrease in stiffness as measured by the Plate Stiffness Test of at least about 50% when compared to a similar unrestretched absorbent layer. In particular aspects of this embodiment, the absorbent layer has a stiffness of about 2 N·mm or less as measured by the Plate Stiffness Test. In another aspect of this embodiment, the absorbent layer exhibits an increase in absorbent capacity index as measured by the Composite Centrifuge Retention Capacity Index Test of at least about 10% when compared to a similar unrestretched absorbent layer. In particular aspects of this embodiment, the absorbent layer has an absorbent capacity index of at least about 7 as measured by the Composite Centrifuge Retention Capacity Index Test. In other particular aspects of this embodiment, the absorbent layer has a thickness of about 2 mm or less. In yet other particular aspects of this embodiment, the absorbent layer has a CD wet strength of at least about 11 N as measured by ASTM D-5034.

The result is an article which exhibits improved performance as well as greater comfort and confidence among the user. In some aspects, the superabsorbent polymer composition can provide fluid adhesive properties in addition to retention properties. Thus, the superabsorbent polymer composition is particularly suitable for use in forming absorbent products and/or laminated products. The term “superabsorbent polymer composition” may also be referred herein as “composition,” as “polymer composition,” or as “absorbent material.”

The articles of the present invention include, for example, personal care absorbent articles, health/medical absorbent articles, or household/industrial absorbent articles. Such articles may further be disposable. Disposable absorbent articles can include a backsheet, an optional topsheet joined to the backsheet, and an absorbent layer positioned and held between the topsheet and the backsheet. The topsheet is operatively permeable to the fluids that are intended to be held or stored by the absorbent article, and the backsheet may or may not be substantially impervious or otherwise operatively impermeable to the intended fluids. The absorbent article may also include other components, such as liquid wicking layers, liquid intake layers, liquid distribution layers, transfer layers, barrier layers, wrapping layers and the like, as well as combinations thereof. Disposable absorbent articles and the components thereof can operate to provide a body-facing surface and an outward-facing surface. As used herein, a body-facing or bodyside surface means that the surface of the article or component which is intended to be disposed toward or placed adjacent to the body of the wearer during ordinary use, while the outward-facing surface is on the opposite side, and is intended to be disposed to face away from the wearer's body during ordinary use. Such outward surface may be arranged to face toward or may be placed adjacent to the wearer's undergarments when the absorbent article is worn.

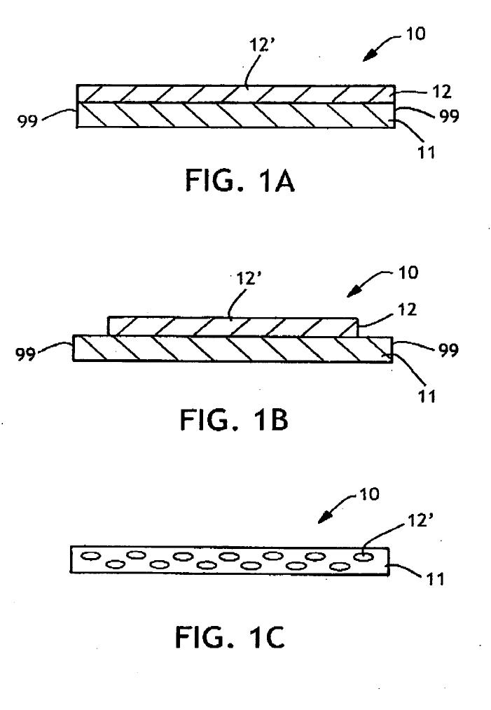

The absorbent layer may comprise the superabsorbent polymer composition alone, or may also include a substrate, as is shown in FIGS. 1A and 1B, which are described in more detail below, or the absorbent material may be impregnated into the substrate, as is shown in FIG. 1C. With reference to FIG. 1A, which shows a cross-section of the absorbent layer of the present invention, the absorbent layer 10 has a support substrate 11 and an absorbent layer 12. In one embodiment of the present invention, the absorbent layer may be formed on a surface of the support substrate from an absorbent material 12′. As will be explained in more detail below, the absorbent material is prepared from a superabsorbent polymer composition.

As is shown in FIG. 1A, the absorbent layer 12 may be coextensive with the substrate 11. However, in the present invention, it is not necessary that the absorbent layer 12 is coextensive with the substrate layer 11. That is, the absorbent layer 12 does not completely cover the substrate 11 to the outer edges 99 of the substrate. In an alternative embodiment of the present invention shown in FIG. 1B, the absorbent layer 12 containing the absorbent material 12′ is not coextensive with the substrate 11, covering only a portion of the substrate 11 short of the outer edges 99 of the substrate 11. In another alternative embodiment of the present invention, the absorbent material may be placed within the substrate or impregnated into the substrate. This is shown in FIG. 1C, where the absorbent material 12′ is placed within the substrate 11. In order for the absorbent material to be impregnated or otherwise placed within the substrate 11, the substrate should be prepared from a material which contains interstitial spaces that allow the absorbent material 12′ to penetrate the surface of the substrate 11 and allow the absorbent material 12′ to be within interstitial spaces within the substrate 11. Whether the absorbent material is a layer on the substrate or placed within the substrate, the substrate can act as a support layer to support the absorbent material, and the absorbent material layer can be prepared on the substrate. It is noted that the absorbent material 12′ may appear to be shown in FIG. 1C as a discrete phase or as discrete particles; however, the intent is to show that the superabsorbent polymer composition 12′ is impregnated into the substrate 11. Thus, the superabsorbent polymer composition could be a continuous phase within the substrate.

The support substrate of the absorbent layer may comprise a wide variety of materials. In addition, the support layer (i.e., the support substrate) can be liquid permeable or liquid impermeable. In some aspects, the support layer can be a film, a nonwoven web, a knitted fabric or a woven fabric, or a laminate of one or more of these materials. The only requirements for the substrate layer is that it has sufficient integrity so that the superabsorbent polymer composition may be placed onto the substrate layer, or in the case of substrates with interstitial spaces, such as knitted fabrics, woven fabrics and nonwoven webs, or laminates containing these substrate materials, can be impregnated with the superabsorbent polymer composition. In particular aspects of the invention, the substrate can be a surge material, such as that described in U.S. Publication No. US 2005/0098256 to Polanco et al., which is hereby incorporated by reference in a manner that is consistent herewith. In addition, the substrate should have sufficient flexibility so that the absorbent can be used in flexible absorbent articles. Particular examples of substrates include, but are not limited to, films, spunbond nonwoven webs and laminates of films and spunbond nonwoven webs, surge materials, bonded-carded-webs, bonded-airlaid webs, coform, and woven fabrics such as cotton and wool cloth.

In general, as the thickness of the substrate increases, the integrity also increases. In some aspects, it may be desirable to have a very thin substrate to help increase flexibility and to help decrease the thickness of an absorbent article. However, if the thickness of the substrate is less that about 0.01 mm, the substrate may be damaged during formation of the absorbent layer or during use in an absorbent article. For example, in some aspects, when the substrate is a nonwoven web, the basis weight can be below about 100 gsm. However, in other aspects, the nonwoven substrate could have a basis weight in excess of 100 gsm. In some aspects, the basis weight of the nonwoven web could be between about 7 gsm and about 60 gsm, such as between about 10 gsm and about 40 gsm. In general, if the basis weight is below about 7 gsm, the nonwoven web will tend to have insufficient strength to support the absorbent material.

In some aspects of the present invention, the superabsorbent polymer composition may be placed directly on the substrate and can be directly joined or directly connected to the substrate, without the addition of adhesives, thereby forming a layer on the substrate. In other aspects, the superabsorbent polymer composition can penetrate the substrate such that it will be impregnated into the support substrate.

The superabsorbent polymer composition may be applied to the substrate using any suitable application process, including slot coating, screen coating, knife over roll coating, or roll coating, either in a continuous coverage or a patterned coverage. Printing applications are other suitable application techniques, including gravure printing, screen, roll, rotary roll and jet printing. The superabsorbent polymer composition may also be applied to the substrate using a spray application. The actual method of application of the superabsorbent polymer composition to the substrate is not critical to the present invention. Once placed on the substrate, the superabsorbent polymer composition is crosslinked, forming an absorbent coating on the substrate or forming a crosslinked absorbent material impregnated within the substrate. In some aspects, the absorbent layer can include at least about 60% by weight of the flexible superabsorbent binder polymer composition, and not more than about 40% by weight of the fibers, based on a total weight of the composite.

One advantage of the superabsorbent polymer composition of the present invention is that it provides a water-soluble ionic polymer capable of sufficient spontaneous crosslinking within about 10 minutes, such as less than about 5 minutes, or less than about 1 minute, at a web temperature not more than about 150° C., to provide the absorbent layer with an absorbent capacity of at least one (1) gram of fluid per gram of superabsorbent polymer composition, such as at least three (3) grams of fluid per gram of superabsorbent polymer composition, using the Centrifuge Retention Capacity Test (described below). In some aspects, the ionic polymer may bear a positive charge, a negative charge, or a combination of both, and should have an ionic unit content of about 15 mole % or greater. The ionic polymer may include a variety of monomer units described below.

Eliminating the need for radiative crosslinking provides a significant processing advantage. The crosslinking at web temperatures not more than about 150° C., such as not more than about 120° C., or not more than about 100° C., permits the superabsorbent polymer composition to be applied to one or more substrate layers, and then crosslinked, without degrading or damaging the substrate. Significant crosslinking occurs within about 10 minutes, such as within about 8 minutes, or within about 6 minutes to provide an efficient, commercially feasible, cost-effective crosslinking process. The crosslinking may then continue until a superabsorbent polymer composition having the desired absorbent capacity or a desired dryness is obtained. A suitable dryness may be at least about 92% by weight solids of the superabsorbent polymer composition. Drying the absorbent layer may be accomplished through conventional means known in the art, for example, a flotation dryer system, to promote the crosslinking, as well as the drying, of the absorbent layer.

While the absorbent layer is being dried and crosslinked, shrinkage in the cross-machine direction (CD), the machine direction (MD) or both occurs as a result of the crosslinking and potential fiber re-orientation within the substrate. It has been discovered that by restretching the absorbent layer (i.e., pulling the shrinkage back out) such as to approximately its original dimensions (i.e., the dimensions prior to crosslinking and drying), the absorbent layer exhibits a “fissuring” effect to the superabsorbent polymer composition of the present invention. As used herein, the term “fissuring” refers to transforming a substantially continuous layer of the dried superabsorbent polymer composition into a substantially discontinuous layer by creating cracks and fissures within the layer. However, the superabsorbent polymer composition remains substantially connected to the substrate. As a result of the fissuring effect, the absorbent layer exhibits a significant improvement in various properties, such as stiffness and absorbent properties, when compared to a similar unrestretched absorbent layer. Therefore, equivalent absorbent properties can be obtained while utilizing a thinner and/or less dense absorbent layer, as compared to similar unrestretched absorbent layers. For example, in some aspects, the absorbent layer can exhibit an increase in absorbent capacity as measured by the Composite Centrifuge Retention Capacity Test of at least about 30% when compared to a similar unrestretched absorbent layer. In particular aspects, the absorbent layer can achieve an absorbent capacity of at least about 800 g/m², such as between about 800 and 1400 g/m² as measured by the Composite Centrifuge Retention Capacity Test. In other aspects, the absorbent layer has a thickness of about 1 mm or less, such as between about 0.05 mm and about 1 mm. In still other aspects, the absorbent layer has a density of about 0.1 g/cc or less, such as between about 0.09 g/cc and about 0.1 g/cc. In yet other aspects, the absorbent layer exhibits a decrease in stiffness as measured by the Plate Stiffness Test of at least about 50% when compared to a similar unrestretched absorbent layer. In some particular aspects, the absorbent layer has a stiffness of about 2 N·mm or less as measured by the Plate Stiffness Test, such as between about 0.3 and 2 N·mm, or between about 0.3 and 1 N·mm as measured by the Plate Stiffness Test. In still other aspects, the absorbent layer can a CD wet tensile of at least about 11 N as measured by ASTM D-5034.

The absorbent layer may also include an additional substrate, which may be positioned such that the superabsorbent polymer composition is positioned between the support layer and the additional substrate. In some aspects, when an additional substrate is included in the absorbent layer, the density of the absorbent layer is desirably less than about 0.3 g/cc, such as between about 0.09 and 0.28 g/cc to provide desired benefits. The thickness of the absorbent layer which comprises an additional substrate is desirably less than about 2 mm, such as between about 0.1 mm and 2 mm.

The additional substrate may comprise a wide variety of materials. In addition, the additional substrate can be liquid permeable or liquid impermeable. In some aspects, the additional substrate can be a film, a nonwoven web, a knitted fabric or a woven fabric, or a laminate of one or more of these materials. In particular aspects of the invention, the additional substrate can be a surge material, such as that described in U.S. Publication No. US 2005/0098256 to Polanco et al., which was previously incorporated by reference in a manner that is consistent herewith. In addition, the additional substrate should have sufficient flexibility so that it can be used in flexible absorbent articles. Particular examples of additional substrates include, but are not limited to, films, spunbond nonwoven webs and laminates of films and spunbond nonwoven webs, surge materials, bonded-carded-webs, bonded-airlaid webs, coform, and woven fabrics such as cotton and wool cloth.

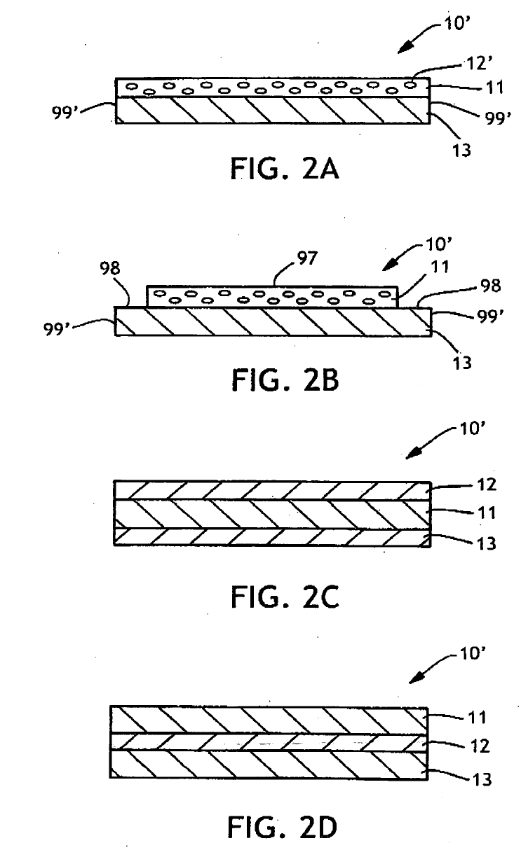

To obtain a better understanding of the absorbent layer with additional substrate layers, attention is directed to FIGS. 2A and 2B. FIG. 2A shows an absorbent layer 10′ having a support substrate layer 11 impregnated with the superabsorbent polymer composition 12′ and an additional substrate 13. The additional substrate 13 is adjacent to the support substrate 11 with the superabsorbent polymer composition 12′ impregnated therein. As is shown in FIG. 2A, the substrate 11 with the superabsorbent polymer composition 12′ impregnated therein is coextensive with the side edges 99′ of the additional substrate layer 13. In an alternative embodiment, shown in FIG. 2B, the support substrate 11 having the superabsorbent polymer composition 12′ applied therein is positioned on the additional substrate 13 such that the support substrate and the superabsorbent polymer composition therein is not coextensive with the edges 99 of the additional substrate. As stated above in regard to FIG. 1C, it is noted that the superabsorbent polymer composition 12′ may appear to be shown in FIGS. 2A and 2B as a discrete phase or as discrete particles. However, the intent is to show that the superabsorbent polymer composition 12′ is impregnated within the substrate 11. That is, the superabsorbent polymer composition 12′ could be a continuous phase within the substrate 11.

In addition, the support substrate 11 may have the superabsorbent polymer composition 12′ and the additional substrate 13 applied to opposing sides of the support substrate 11, as is shown in FIG. 2C. The additional substrate layer 13 may be bonded to the support substrate layer 11 using a known technique, such as adhesive bonding, pattern bonding using heat and pressure, ultrasonic bonding, stitching and other similar joining techniques. The layers of the absorbent layer may be held together using suitable bonding techniques, including those described above. In another aspect, the absorbent material 12 from the superabsorbent polymer composition may adhesively hold the additional substrate layer 13 to the support substrate 11 as is shown in FIG. 2D. When a three layer structure absorbent layer is desired or prepared, the superabsorbent polymer composition may be applied to one of layers 11 or 13 or both layers 11 and 13. The layers are brought together so that the superabsorbent polymer composition contacts each layer of the support substrate 11 and additional substrate 13 of the absorbent layer. As a result, the superabsorbent polymer composition and the resulting absorbent layer 12 are directly joined to the adjacent support substrate 11 and additional substrate 13, without an additional adhesive. This may be accomplished by applying the superabsorbent polymer composition to facing surfaces of one or both layers 11 and 13, bringing the layers 11 and 13 together so that the superabsorbent polymer composition contacts both layers, and crosslinking the superabsorbent polymer composition to form the absorbent layer 12. In some aspects, crosslinking can be moisture-induced by hydrolysis and condensation of alkoxysilanes. For example, crosslinking of the superabsorbent polymer composition can be induced by concentrating the composition through the removal of the water to promote condensation of silanols generated by hydrolysis of alkoxysilanes.

The superabsorbent polymer composition layer may be formed on the support substrate as a continuous layer having uniform thickness, or as a discontinuous or nonuniform layer which provides flow channels, liquid retention dams, or other desired attributes. However, because the absorbent layer 12 is intended as a sole or primary absorbent layer in the simplified absorbent article, the superabsorbent polymer composition should be present in sufficient thickness and quantity, and over a sufficient area, to provide substantially all of the liquid absorption capacity that is required by the end use application. Alternatively, in some aspects, superabsorbent materials, such as superabsorbent particles, can be additionally incorporated into the absorbent to provide a portion of the liquid absorption capacity required by the end use application.

Because the superabsorbent polymer composition is in contact with layers 11 and 13 as it is being formed, the resulting absorbent layer 12 adheres to the support substrate layer and the additional substrate layer 13 in addition to serving as an absorbent (fluid storage) layer. Thus, in some aspects of the present invention, the absorbent layer 10′ can provide three layers bound together in sequence (i.e., a fluid receiving layer or backsheet, an absorbent layer, and a support layer) without intervening adhesive layers. The resulting absorbent layer can then be heated and restretched using the means discussed above to fissure the dry, crosslinked superabsorbent polymer composition.

In other aspects, the superabsorbent polymer composition may be prepared using a continuous process wherein the polymerization and/or neutralization reaction is carried out in a suitable reactor that conveys the resulting superabsorbent polymer composition, upon completion of the polymerization reaction, directly to an apparatus for applying the composition onto the support substrate layer 11 and/or the additional substrate layer 13. Suitable apparatus are discussed above. Such a continuous process may be desirable where conditions, such as high heat, can cause premature crosslinking of the superabsorbent polymer composition that would hinder application of the composition onto the substrate. The thickness of the absorbent layer of this embodiment is substantially determined by the thickness of the support layer and of the additional layer. However, it is desirable that the thickness is less than about 2 mm, such as less than about 1 mm, or between about 0.05 mm and about 1 mm.

In other aspects of the present invention, the additional substrate forms a backsheet for an absorbent article. The backsheet serves to prevent any fluids absorbed by the substrate 11 and the superabsorbent polymer composition 12′ applied thereon from passing through the absorbent article. Generally, the backsheet is fluid impermeable. In yet other aspects of the present invention, a second additional substrate serves as a topsheet for the absorbent article. The topsheet protects the support substrate and the superabsorbent polymer composition applied thereon during use of the absorbent article. In addition, the topsheet may serve to protect the user of the absorbent article from having direct contact with any additional superabsorbent particles that may optionally be present in the superabsorbent polymer composition 12′.

In still other aspects of the present invention, the absorbent layer 10′ may have two distinct areas of the layer. To obtain a better understanding of this aspect of the present invention, attention is again directed to FIG. 2B, which shows an absorbent layer 10′ having a central region 97 and a perimeter region 98. The central region includes both the additional substrate 13, and the support substrate 11 and the superabsorbent polymer composition 12′ applied thereon adjacent to the additional substrate 13 to form an absorbent article. The perimeter region 98 only includes the additional substrate 13 or backsheet. In some aspects, this area is located in an insult target zone.

In some aspects, the absorbent layer of the present invention can be relatively thin and can have a thickness in the range of about 1 mm or less, such as about 0.05 mm to about 1 mm at a pressure of 1.35 kPa when only one substrate is present. In other aspects, the absorbent layer of the present invention can have a thickness in the range of about 2 mm or less, such as about 0.05 mm to about 2 mm, or about 0.05 mm to about 1 mm at a pressure of 1.35 kPa when both a support substrate and an additional substrate are present. Generally, it is desirable that the absorbent layer be as thin as possible while providing sufficient absorbency. In some aspects, the absorbent layer of the present invention has a thickness in the range of about 0.1 mm to about 1.0 mm, such as about 0.2 to about 0.9 mm. In addition, the superabsorbent polymer composition of the present invention can have an absorbency greater than 1 gram of 0.9% NaCl solution per gram of superabsorbent polymer composition (g/g), such as up to about 10 g/g or greater, or about 1 g/g to about 5 g/g, as measured by the Centrifuge Retention Capacity Test.

In one aspect of this embodiment, the absorbent layer comprising an additional layer has a density of about 0.3 g/cc or less. In other aspects of this embodiment, the absorbent layer exhibits a decrease in stiffness as measured by the Plate Stiffness Test of at least about 50% when compared to a similar unrestretched absorbent layer. In particular aspects of this embodiment, the absorbent layer has a stiffness of about 2 N·mm or less as measured by the Plate Stiffness Test. In another aspect of this embodiment, the absorbent layer exhibits an increase in absorbent capacity index as measured by the Composite Centrifuge Retention Capacity Index Test of at least about 10% when compared to a similar unrestretched absorbent layer. In particular aspects of this embodiment, the absorbent layer has an absorbent capacity index of at least about 7 as measured by the Composite Centrifuge Retention Capacity Index Test. In yet other particular aspects of this embodiment, the absorbent layer has a CD wet strength of at least about 11 N as measured by ASTM D-5034.

The absorbent layer of the present invention can be used on its own or as an absorbent component in a wide variety of absorbent articles including, but not limited to, personal care absorbent articles, household/industrial absorbent articles and health/medical absorbent articles. In some aspects, the absorbent layer of the present invention may be particularly suited for use in sanitary napkins, pantiliners, bandages, bed liners, furniture pads, as well as other absorbent articles, as referenced above. Typically, absorbent articles have an absorbent layer, and a backsheet which helps retain any absorbed fluids in the absorbent article. Most absorbent articles have a backsheet which is a liquid impermeable layer. The backsheet generally faces away from the fluid source, meaning that the absorbent layer is positioned between the fluid source and the backsheet. In some applications, such as a bandage, the backsheet may be an apertured material, such as an apertured film, or material which is otherwise gas permeable, such as gas permeable films. In absorbent personal care articles such as pantiliners, the backsheet which is a liquid impermeable layer is often a garment facing layer. The backsheet is often referred to as a backing layer, baffle or outercover. Additional layers, such as a topsheet, also commonly referred to as a bodyside liner, may also be present in the absorbent article of the present invention.

As referenced above, the absorbent layer includes a desired amount of the superabsorbent polymer composition of the present invention. In some aspects, the superabsorbent polymer composition includes about 15 to about 99.8% by mass of monoethylenically unsaturated polymer units, such as about 25 to about 89.5% by mass, or about 30 to about 79% by mass, or about 50 to about 70% by mass. Suitable monoethylenically unsaturated polymer units include, without limitation, monoethylenically unsaturated carboxylic acid units and salts thereof, monoethylenically unsaturated sulphonic acid units and salts thereof, and monoethylenically unsaturated phosphonic acid units and salts thereof. Suitable monoethylenically unsaturated monomers that can be used to form the monoethylenically unsaturated polymer units include, without limitation:

a) Carboxyl group-containing monomers including monoethylenically unsaturated mono or poly-carboxylic acids, such as (meth)acrylic acid (meaning acrylic acid or methacrylic acid; similar notations are used hereinafter), maleic acid, fumaric acid, crotonic acid, sorbic acid, itaconic acid, and cinnamic acid;

b) Carboxylic acid anhydride group-containing monomers, including monoethylenically unsaturated polycarboxylic acid anhydrides (such as maleic anhydride);

c) Carboxylic acid salt group-containing monomers including water-soluble salts (alkali metal salts, ammonium salts, amine salts, etc.) of monoethylenically unsaturated mono- or poly-carboxylic acids (such as sodium (meth)acrylate, trimethylamine (meth)acrylate, triethanolamine (meth)acrylate), sodium maleate, methylamine maleate;

d) Sulfonic acid group-containing monomers, including aliphatic or aromatic vinyl sulfonic acids (such as vinylsulfonic acid, allyl sulfonic acid, vinyltoluenesulfonic acid, stryrene sulfonic acid), (meth)acrylic sulfonic acids [such as sulfopropyl (meth)acrylate, 2-hydroxy-3-(meth)acryloxy propyl sulfonic acid];

e) Sulfonic acid salt group-containing monomers, including alkali metal salts, ammonium salts, amine salts of sulfonic acid group containing monomers as mentioned above; and/or f) Amide group-containing monomers, including vinylformamide, (meth)acrylamide, N-alkyl (meth)acrylamides (such as N-methylacrylamide, N-hexylacrylamide), N,N-dialkyl (meth)acryl amides (such as N,N-dimethylacrylamide, N,N-di-n-propylacrylamide), N-hydroxyalkyl (meth)acrylamides [such as N-methylol (meth)acrylamide, N-hydroxyethyl (meth)acrylamide], N,N-dihydroxyalkyl (meth)acrylamides [such as N,N-dihydroxyethyl (meth)acrylamide], 3-acrylamidopropyl trimethyl ammonium chloride, vinyl lactams (such as N-vinylpyrrolidone).
The superabsorbent polymer composition can also include about 0.1 to about 20% by mass of polyacrylate ester units, such as acrylate and/or methacrylate ester units, that include an alkoxysilane functionality. The acrylate and/or methacrylate ester units are copolymerized with the monoethylenically unsaturated monomer units. In particular, the superabsorbent polymer composition may include about 0.5 to about 15% by mass of the acrylate and/or methacrylate ester units, for instance about 1.0 to about 10% by mass, for instance about 1.5 to about 5.5% by mass.

The alkoxysilane functionality is a functional group or moiety that reacts with water to form a silanol group. One suitable alkoxysilane group is a trialkoxy silane group having the following structure: 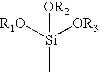
wherein R₁, R₂ and R₃ are alkyl groups independently having from 1 to 6 carbon atoms.

The term “monomer(s)” as used herein includes monomers, oligomers, polymers, mixtures of monomers, oligomers and/or polymers, and any other reactive chemical species which is capable of co-polymerization with monoethylenically unsaturated carboxylic, sulphonic or phosphoric acid or salts thereof. Ethylenically unsaturated monomers containing a trialkoxy silane functional group are appropriate for this invention and are desired. Suitable ethylenically unsaturated monomers include acrylates and methacrylates. A particularly ethylenically unsaturated monomer containing a trialkoxy silane functional group is methacryloxypropyl trimethoxy silane, commercially available from Dow Corning (having a place of business in Midland, Mich., U.S.A.) under the trade designation Z-6030 Silane. Other suitable ethylenically unsaturated monomers containing a trialkoxy silane functional group include, but are not limited to, methacryloxyethyl trimethoxy silane, methacryloxypropyl triethoxy silane, methacryloxypropyl tripropoxy silane, acryloxypropylmethyl dimethoxy silane, 3-acryloxypropyl trimethoxy silane, 3-methacryloxypropylmethyl diethoxy silane, 3-methacryloxypropylmethyl dimethoxy silane, and 3-methacryloxypropyl tris(methoxyethoxy) silane. However, it is contemplated that a wide range of vinyl and acrylic monomers having trialkoxy silane functional groups or a moiety that reacts easily with water to form a silanol group, such as a chlorosilane or an acetoxysilane, provide the desired effects and are effective monomers for copolymerization in accordance with the present invention.

The superabsorbent polymer composition also includes zero to about 75% by mass polyolefin glycol and/or polyolefin oxide units, such as about 0.1 to about 75% by mass polyolefin glycol and/or polyolefin oxide units, or about 5 to about 75% by mass, or about 10 to about 60% by mass, or about 20 to about 50% by mass, or about 30 to about 40% by mass. The polyolefin glycol or oxide may be a glycol or oxide of an olefin polymer having about 2 to about 4 carbon atoms. Polyethylene glycol, polyethylene oxide, polypropylene glycol and polypropylene oxide are examples of suitable polymer units. The polyolefin glycol and/or polyolefin oxide may include on average about 30 to about 15,000 glycol and/or oxide units per molecule. The weight average molecular weight of polyolefin glycol units may range from about 200 to about 8000. When polyolefin oxide units are employed, they may have a weight average molecular weight of about 100,000 to about 600,000.

Polyolefin glycols and polyolefin oxides are commercially available, and are common. To prepare the superabsorbent polymer composition of the invention, a pre-formed polyolefin glycol and/or oxide may be dissolved or dispersed in a reaction vessel which includes an aqueous solvent or carrier, an organic solvent or carrier such as ethanol, or a miscible combination of aqueous and organic solvent or carrier. The monomers used to form the monoethylenically unsaturated polymer units and the polyacrylate ester units are added to the solution and polymerized using a template polymerization process in which the polyolefin glycol or oxide serves as a template polymer. Before initiation, the polar groups of the monomers, for instance the acid groups of acrylic acid, are attracted to the polyolefin glycol and/or polyolefin oxide through hydrogen bonding. The steric alignment of the monomers, with the polyolefin glycol and/or oxide serving as backbone, aids in the polymerization and typically increases the chain length of the polymerizing unit. During the polymerization, radical polymerizing chains may become attached to the template polymer, resulting in grafting of polyolefin glycol and/or oxide to the copolymer being formed. However, this graft polymerization need not occur. The resulting superabsorbent polymer composition includes the polyolefin glycol and/or oxide attached to, and/or blended with, the copolymer of the monoethylenically unsaturated polymer units and the acrylate or methacrylate ester units that include the alkoxysilane functionality.

The polymerization may be initiated using a variety of methods, including without limitation thermal energy, ultraviolet light, and redox chemical reactions. Desirably, redox initiation can be used for the polymerization. This method incorporates a first monomer solution that includes a reducing polymerization initiator. Suitable reducing polymerization initiators include, but are not limited to, ascorbic acid, alkali metal sulfites, alkali metal bisulfites, ammonium sulfite, ammonium bisulfite, alkali metal hydrogen sulfites, ferrous metal salts such as ferrous sulfates, sugars, aldehydes, primary and secondary alcohols, and combinations thereof. In one embodiment, the reducing polymerization initiator includes ascorbic acid.

The second monomer solution further includes an oxidizing polymerization initiator. Suitable oxidizing initiators include, but are not limited to, hydrogen peroxide, alkali metal persulfates, ammonium persulfate, alkylhydroperoxides, peresters, diacryl peroxides, silver salts, and combinations thereof. In one embodiment, the oxidizing polymerization initiator includes hydrogen peroxide.

Generally, when the first aqueous monomer solution is combined with the second aqueous monomer solution the reducing polymerization initiator reacts with the oxidizing polymerization initiator (e.g., a redox reaction) thereby initiating a polymerization reaction to form a binder composition including a monoethylenically unsaturated monomer and an ethylenically unsaturated monomer that has post-application, moisture-induced crosslinking capability.

In some aspects, the monoethylenically unsaturated polymer unit is a cationic polymer. The cationic polymer is advantageous because it provides: a) inherent antimicrobial properties; b) enhanced attraction and retention into cellulose fibers in a suspension; and c) enhanced attraction to superabsorbent particles which are negatively charged. Suitable cationic polymers include those prepared by copolymerizing a monomer 1) selected from: a) acryloyloxyethyl-trialkyl-substituted ammonium salts; b) acryloyloxypropyl-trialkyl-substituted ammonium salts; c) acrylamidoethyl-trialkyl-substituted ammonium salts; and d) acrylamidopropyl-trialkyl-substituted ammonium salts, with a monomer 2) selected from: a) methacryl esters which contain an alkoxysilane group capable of moisture-induced crosslinking and b) acryl esters which contain an alkoxysilane group capable of moisture-induced crosslinking. Other monomers may also be present, for instance, an acrylic acid or acrylamide. The polymerization is conducted in the presence of a polyolefin glycol and/or polyolefin oxide as described above, suitably a polyethylene glycol. The cationic monoethylenically unsaturated monomer unit and the polyolefin glycol are present in the amounts described above.

Once the cationic monoethylenically unsaturated polymer has been prepared (e.g., by a redox initiation process), the cationic copolymer is then coated and dried onto a substrate to form the crosslinked absorbent coating.

The substrate layers (i.e., the support substrate and/or the additional substrate) of the present invention can be extensible or can be elastically extensible. In some aspects, the support substrate can include an operative amount of fluff and/or elastomeric polymer fibers. For example, fibers can be “crimped” and then the substrate can be extended by pulling out the crimp. Also, substrates without elastomeric fibers can shrink, as described above, or become more narrow in the lateral direction when heated under tension as a result of crosslinking and/or potentially more alignment of the fibers. This “necked” fabric can be restretched (i.e., extended again) by pulling in the lateral direction.

Suitable restretching techniques are known in the art. For example, one suitable method can be found in U.S. Pat. No. 4,285,100 to Schwarz, which is hereby incorporated by reference in a manner that is consistent herewith. Schwarz describes a process and apparatus for selectively stretching a web in a station provided with a set of grooved rolls. The groove pattern of the rolls is generally a sinosoidal wave having a distance between grooves of less than 1.0 millimeter times the web basis weight in grams per square meter. The web is stretched in a manner to affect uniform stretching to produce a web of larger dimension in the direction of stretch and a correspondingly fissured superabsorbent polymer layer. Shwarz also provides a method and apparatus for bi-axially stretching a web in a first and second station where the first and second stations are provided with sets of rolls having grooves parallel and perpendicular, respectively, to the axis of each set of rolls. The groove pattern of each set of rolls is such that the distance between grooves is less than 1.0 millimeter times the web basis weight in grams per square meter. The web is stretched in a manner to affect uniform stretching to a bi-axially larger dimension and which correspondingly fissures the superabsorbent polymer composition layer.

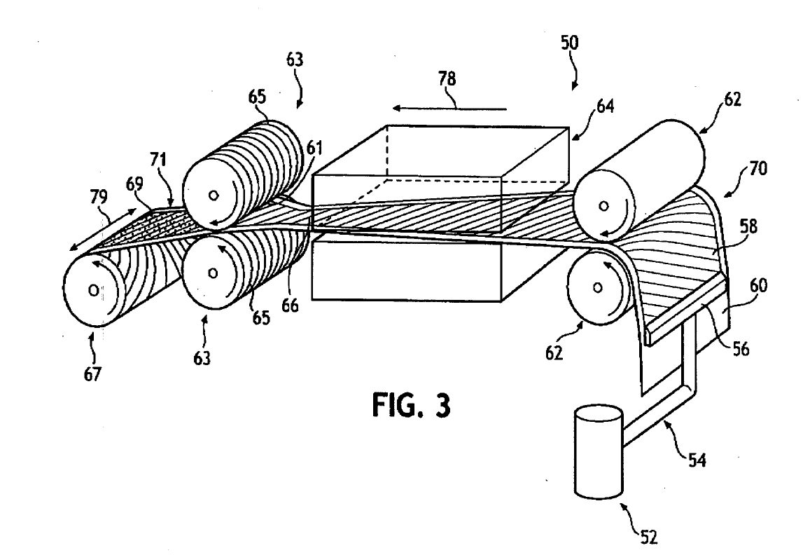

A process to manufacture the absorbent layer of the present invention can be described with reference to FIG. 3. Provided is an restretched absorbent layer producing machine 50. Uncrosslinked superabsorbent polymer composition is pumped from a drum 52 through a pipe 54 to a slot die 56. The uncrosslinked superabsorbent polymer composition is then deposited as a layer 58 onto or into a support substrate 60 to form an absorbent layer 70. The absorbent layer 70 travels through a set of rolls 62 and into a flotation dryer 64 which heats the absorbent layer 70 to a temperature of between about 100° C. and 150° C., which causes shrinking of the absorbent layer 70 in the cross-machine direction, in the machine direction, or both due to crosslinking of the superabsorbent polymer composition 58 and/or fiber re-orientation of the substrate 60 to form a necked absorbent layer 66. The necked absorbent layer 66 then passes between the nip 61 of a pair of rollers 63 having a plurality of grooves 65 parallel to the axis of the rollers 63. The grooves 65 of the rollers 63 are intermeshed like gears, as known to those skilled in the art. As the necked absorbent layer 66 enters the nip 61, the necked absorbent layer 66 assumes the shape of a grove 65 and is stretched in at least the cross-machine direction 79 to form a restretched absorbent layer 71 in which the superabsorbent polymer material has been fissured 69. The restretched absorbent layer 71 is then wound onto a reel 67. In addition, the speed of the reel 67 may be greater than the speed of the incoming absorbent layer 71 such that restretching also occurs in the machine direction 78.

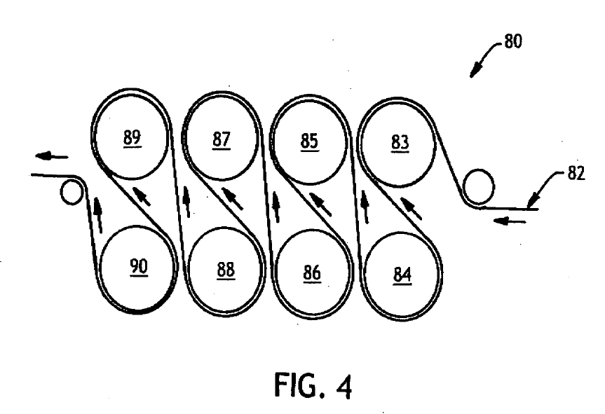

By way of example only, an alternative means for restretching the absorbent layer can be described with reference to FIG. 4. Provided is a progressive draw steam can dryer system 80. In this system, each dryer can 83-90 turns at a rate that is higher than the previous can. As the absorbent layer 82 passes over each can, the faster speed of each progressive can in comparison to the previous can has the effect of restretching the absorbent layer in the machine direction. In addition, the cans 83-90 could be grooved (not shown) in such a manner that restretching in the cross-machine direction occurs. The result is a fissuring 68 of the dry, crosslinked polymer composition.

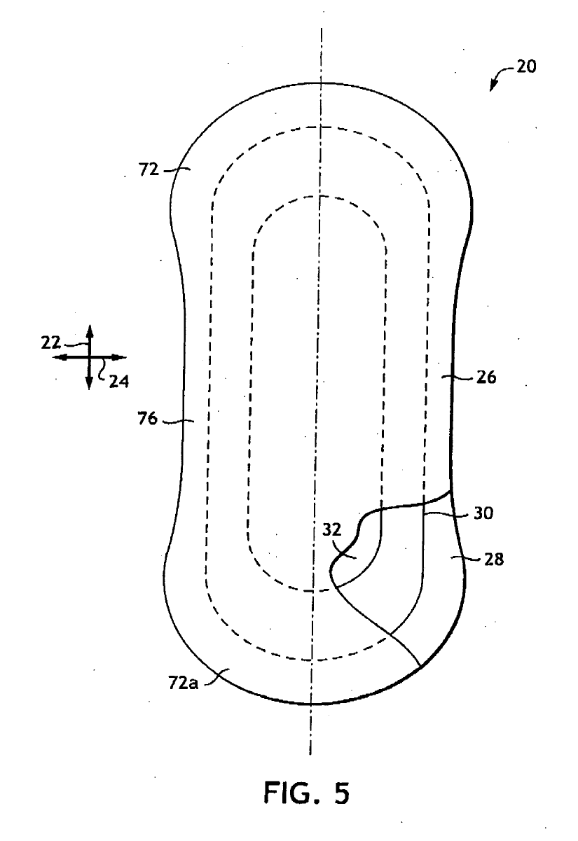

In the present invention, the absorbent layer of the absorbent article can be prepared as described above. As discussed, the absorbent layer includes a support substrate having the superabsorbent polymer composition of the present invention applied thereto, or it may also have an additional substrate. For example, if the support substrate is a liquid impermeable material, the substrate layer of the absorbent layer could also function as the liquid impermeable layer of the absorbent article, for example, the backsheet of the absorbent article. To obtain a better understanding of an absorbent article of the present invention, attention is directed to FIG. 5.

FIG. 5 illustrates an example of a suitable article, such as the representatively shown feminine care article 20, which is configured to incorporate the present invention. It is understood that the present invention is suitable for use with various other articles, including but not limited to other personal care articles, health/medical articles, household/industrial articles, and the like, without departing from the scope of the present invention.

The article can comprise an absorbent body structure, and the absorbent body can include the absorbent layer 30 of the present invention only. In some aspects, the absorbent layer 30 may also include additional components, such as wood fibers or other superabsorbent materials. For example, the absorbent layer 30 may comprise the superabsorbent polymer composition of the present invention operatively contained within or in contact with a matrix of fibers.

The substrate may include an operative amount of polymer fibers and optionally fluff. In some aspects, the layer 30 can include at least about 60% by weight of the fissured superabsorbent polymer composition, and not more than about 40% by weight of the fibers, based on a total weight of the composite.

The article 20 can have a lengthwise longitudinal direction 22, a transverse, laterally extending, cross-direction 24, first and second longitudinally opposed end portions 72 and 72a, and an intermediate portion 76 located between the end portions. As representatively shown, the longitudinal dimension of the article is relatively larger than the lateral dimension of the article. The article 20 can include a topsheet 26, a backsheet 28, and the absorbent layer 30 positioned between the topsheet and backsheet. The absorbent layer 30 can have configurations which are selectively constructed and arranged to provide desired performance and aesthetics.

By incorporating its various features, aspects and configurations, alone or in desired combinations, the article can provide an improved absorbent system that can take better advantage of the functional properties of the superabsorbent polymer composition of the present invention. For example, the article can provide comparable or improved absorbent properties when compared to an article comprising a conventional absorbent layer. Similarly, the article can provide comparable or improved absorbent properties while reducing the thickness and/or improving the flexibility of the article, as well as improved production costs, when compared to an article comprising a conventional absorbent layer.

In some aspects, the absorbent layer 30 can also distribute viscous fluids more efficiently to desired locations in an absorbent article, as well as provide a drier body-facing surface, and in particular configurations, can provide visual cues of absorbency. Other examples can provide improved appearance and aesthetics. As a result, an article incorporating the invention can provide greater comfort and fit, and can improve protection and increase confidence.

The optional topsheet 26 of the article 20 may include a layer constructed of any operative material, and may be a composite material. For example, the topsheet layer can include a woven fabric, a nonwoven fabric, a polymer film, a film-fabric laminate or the like, as well as combinations thereof. Examples of a nonwoven fabric include spunbond fabric, meltblown fabric, coform fabric, carded-web, bonded-carded-web, bicomponent spunbond fabric or the like, as well as combinations thereof. Other examples of suitable materials for constructing the topsheet layer can include rayon, bonded-carded-webs of polyester, polypropylene, polyethylene, nylon, or other heat-bondable fibers, polyolefins, such as copolymers of polypropylene and polyethylene, linear low-density polyethylene, biodegradable aliphatic polyesters such as poly(hydroxyl alkanoates) and polyactic acid, finely perforated film webs, net materials, and the like, as well as combinations thereof.

A more particular example of a suitable topsheet layer material can include a bonded-carded-web composed of polypropylene and polyethylene, such as has been used as a topsheet stock for KOTEX brand pantiliners, and has been obtainable from Vliesstoffwerk Christian Heinrich Sandler GmbH & Co. KG, a business having an address at Postfach 1144, D95120 Schwarzenbach/Saale, Germany. Other examples of suitable materials are composite materials of a polymer and a nonwoven fabric material. The composite materials are typically in the form of integral sheets generally formed by the extrusion of a polymer onto a web of spunbond material. In a desired arrangement, the topsheet layer 26 can be configured to be operatively fluid-permeable with regard to the fluids that the article is intended to absorb or otherwise handle. The operative fluid-permeability may, for example, be provided by a plurality of pores, apertures or other openings, as well as combinations thereof, that are present or formed in the topsheet layer. The apertures or other openings can help increase the rate at which bodily fluids can move through the thickness of the topsheet layer and penetrate into the other components of the article (e.g., the absorbent layer 30). The selected arrangement of liquid-permeability is suitably present at least on an operative portion of the topsheet layer that is appointed for placement on the body-side of the article. The topsheet 26 can provide comfort and conformability, and can function to direct complex fluids, such as menses, away from the body and toward the absorbent layer 30. In one example, the topsheet layer 26 can be configured to retain little or no liquid in its structure, and can be configured to provide a relatively comfortable and non-irritating surface next to the body-tissues of the wearer. The topsheet layer 26 can be constructed of any material which is also easily penetrated by viscous or complex fluids that contact the surface of the topsheet layer.

The optional topsheet 26 can have at least a portion of its bodyside surface treated with a surfactant to render the topsheet more hydrophilic. The surfactant can permit arriving viscous liquids to more readily penetrate the topsheet layer. The surfactant may also diminish the likelihood that the arriving fluids will flow off the topsheet layer rather than penetrate through the topsheet layer into other components of the article. In one example, the surfactant can be substantially evenly distributed across at least a portion of the upper, bodyside surface of the topsheet 26 that overlays the body-facing surface of the absorbent layer 30.

The optional topsheet 26 may be maintained in secured relation with the absorbent layer 30 of the present invention by bonding all or a portion of the adjacent surfaces to a surface of the absorbent layer 30, the backsheet 28, or both. A variety of bonding techniques known to one of skill in the art may be utilized to achieve any such secured relation. Examples of such techniques include, but are not limited to, the application of adhesives in a variety of patterns between the two adjoining surfaces, entangling at least portions of the adjacent surface of the absorbent layer 30 and/or backsheet 28 with portions of the adjacent surface of the topsheet 26, co-aperturing or fusing at least portions of the adjacent surface of the topsheet 26 to portions of the adjacent surface of the absorbent layer 30 and/or backsheet 28. The topsheet 26 may also be held in secured relation to the absorbent layer 30 through adhesive properties exhibited by the superabsorbent polymer composition of the present invention.

The optional topsheet 26 typically extends over the body-facing surface of the absorbent layer 30, but can alternatively extend around the article to partially, or entirely, surround or enclose the composite. Alternatively, the topsheet 26 and the backsheet 28 can have peripheral margins which extend outwardly beyond the terminal, peripheral edges of the absorbent layer 30, and the extending margins can be joined together to partially, or entirely, surround or enclose the composite. In some aspects, the superabsorbent polymer composition of the present invention can function as the topsheet, in addition to functioning as or in an absorbent layer. Thus the superabsorbent polymer composition can also provide an innermost bodyside surface.

The backsheet 28 may include a layer constructed of any operative material, and may or may not have a selected level of liquid-permeability or liquid-impermeability, as desired. In one example, the backsheet 28 may be configured to provide an operatively liquid-impermeable structure. The backsheet 28 may, for example, include a polymeric film, a woven fabric, a nonwoven fabric or the like, as well as combinations or composites thereof. For example, the backsheet 28 may include a polymer film laminated to a woven or nonwoven fabric. In some aspects, the polymer film can be composed of polyethylene, polypropylene, polyester or the like, as well as combinations thereof. Additionally, the polymer film may be micro-embossed, have a printed design, have a printed message to the consumer, and/or may be at least partially colored. Suitably, the backsheet 28 can operatively permit a sufficient passage of air and moisture vapor out of the article, particularly out of the absorbent layer 30, while blocking the passage of bodily fluids.

An example of a suitable backsheet material can include a breathable, microporous film, such as a HANJIN Breathable Backsheet available from Hanjin Printing, Hanjin P&C Company Limited, a business having offices located in Sahvon-li.Jungan-mvu.Kongiu-City, Chung cheong nam-do, Republic of South Korea. This backsheet material is a breathable film, which is white in color, dimple embossed, and contains: 47.78% calcium carbonate, 2.22% TiO2, and 30% polyethylene.

In one example, the polymer film can have a minimum thickness of no less than about 0.025 mm, and in another feature, the polymer film can have a maximum thickness of no greater than about 0.13 mm. Bicomponent films or other multi-component films can also be used, as well as woven and/or nonwoven fabrics which have been treated to render them operatively liquid-impermeable. Another suitable backsheet material can include a closed-cell polyolefin foam. For example, a closed-cell polyethylene foam may be employed. Still another example of a backsheet material would be a material that is similar to a polyethylene film which is used on currently commercially sold KOTEX brand pantiliners, and is obtainable from Pliant Corporation, having a place of business in Schaumburg, Ill., U.S.A. In some aspects, the superabsorbent polymer composition of the present invention can function as the backsheet, in addition to functioning as or in an absorbent layer. Thus, the superabsorbent polymer composition can provide an outermost garment facing surface.

The absorbent layer 30 of the present invention can be sized and placed to more effectively operate in an insult target zone of the absorbent article 20 where liquids are more likely to be introduced into the article. The structure of the absorbent layer 30 can be operatively configured to provide a desired level of fluid acquisition, distribution and retention. The absorbent layer 30 may include one or more components that can modify the composition or rheological properties of such viscous fluids. In some aspects, the absorbent layer 30 may additionally or alternatively include materials such as surfactants, ion exchange resin particles, moisturizers, emollients, perfumes, natural fibers, synthetic fibers, fluid modifiers, odor control additives, and combinations thereof.

In order to function well, the absorbent layer 30 can have certain desired properties to provide improved performance as well as greater comfort and confidence among the user. For instance, the absorbent layer 30 can have corresponding configurations of absorbent capacities, densities, basis weights and/or sizes which are selectively constructed and arranged to provide desired combinations of absorbency properties such as liquid intake rate, absorbent capacity, liquid distribution or fit properties such as stiffness, shape maintenance and aesthetics. Likewise, the components can have desired wet to dry strength ratios, mean flow pore sizes, permeabilities and elongation values.

As mentioned above, the absorbent layer 30 can optionally include a surge material, such as that described in US 2005/0098256 to Polanco et al., previously incorporated by reference in a manner that is consistent herewith. This surge material utilizes the natural crimping ability of certain bicomponent, substantially continuous, thermoplastic fibers of A/B morphology, i.e., a bilateral configuration, generally side by side or eccentric sheath/core construction, to produce high loft, low density nonwoven webs. The fibers are then crimped after filament formation into high loft, low density fabrics. The material may comprise a high loft, low density nonwoven web having a web of substantially continuous, spunbond, helically crimped, bicomponent fibers of A/B morphology. Within the web the fibers are randomly crimped to produce a lofted material with heterogeneous, random, fiber orientation, including heterogeneous z-direction orientation to produce loft of the web, and irregularly spaced openings between the crimped fibers. For example, the material may have a basis weight from about 15 gsm to about 90 gsm exhibiting densities from about 0.002 g/cc to 0.05 g/cc. Alternatively, the fibers may be randomly crimped to produce a lofted material with a very high loft by inducing shingled layers with a buckled z-direction orientation to produce loft of the web, and irregularly spaced openings between the crimped fibers.

In some aspects, the absorbent layer 30 can optionally include elastomeric polymer fibers. The elastomeric material of the polymer fibers may include an olefin elastomer or a non-olefin elastomer, as desired. For example, the elastomeric fibers can include olefinic copolymers, polyethylene elastomers, polypropylene elastomers, polyester elastomers, polyisoprene, cross-linked polybutadiene, diblock, triblock, tetrablock, or other multi-block thermoplastic elastomeric and/or flexible copolymers such as block copolymers including hydrogenated butadiene-isoprene-butadiene block copolymers; stereoblock polypropylenes; graft copolymers, including ethylene-propylene-diene terpolymer or ethylene-propylene-diene monomer (EPDM) rubber, ethylene-propylene random copolymers (EPM), ethylene propylene rubbers (EPR), ethylene vinyl acetate (EVA), and ethylene-methyl acrylate (EMA); and styrenic block copolymers including diblock and triblock copolymers such as styrene-isoprene-styrene (SIS), styrene-butadiene-styrene (SBS), styrene-isoprene-butadiene-styrene (SIBS), styrene-ethylene/butylene-styrene (SEBS), or styrene-ethylene/propylene-styrene (SEPS), which may be obtained from Kraton Inc. (a business having offices located in Houston, Tex., U.S.A.) under the trade designation KRATON elastomeric resin or from Dexco, a division of ExxonMobil Chemical Company (a business having offices located in Houston, Tex. U.S.A.) under the trade designation VECTOR (SIS and SBS polymers); blends of thermoplastic elastomers with dynamic vulcanized elastomer-thermoplastic blends; thermoplastic polyether ester elastomers; ionomeric thermoplastic elastomers; thermoplastic elastic polyurethanes, including those available from Invista Corporation (a business having offices located in Wichita, Kans., U.S.A.) under the trade name LYCRA polyurethane, and ESTANE available from Noveon, Inc. (a business having offices located in Cleveland, Ohio U.S.A.); thermoplastic elastic polyamides, including polyether block amides available from AtoFina Chemicals, Inc. (a business having offices located in Philadelphia, Pa. U.S.A.) under the trade name PEBAX; polyether block amide; thermoplastic elastic polyesters, including those available from E.I. Du Pont de Nemours Co., under the trade name HYTREL, and ARNITEL from DSM Engineering Plastics (a business having offices located in Evansville, Ind., U.S.A.) and single-site or metallocene-catalyzed polyolefins having a density of less than about 0.89 grams/cubic centimeter, available from Dow Chemical Co. (a business having offices located in Freeport, Tex. U.S.A.) under the trade name AFFINITY; and combinations thereof.

The substrate of the absorbent layer 30 can include an amount of a surfactant. The surfactant can be combined with the substrate of the absorbent layer in any operative manner. Various techniques for combining the surfactant are conventional and well known to persons skilled in the art. For example, the surfactant may be compounded with polymer employed to form a meltblown fiber structure. In a particular feature, the surfactant may be configured to operatively migrate or segregate to the outer surface of the fibers upon the cooling of the fibers. Alternatively, the surfactant may be applied to or otherwise combined with spunbond fibers after the fibers have been formed.

The substrate can include an operative amount of surfactant, based on the total weight of the substrate and surfactant. In some aspects, the substrate can include at least a minimum of about 0.1% by weight surfactant, as determined by water extraction. The amount of surfactant can alternatively be at least about 0.15% by weight, and can optionally be at least about 0.2% by weight to provide desired benefits. In other aspects, the amount of surfactant can be generally not more than a maximum of about 2% by weight, such as not more than about 1% by weight, or not more than about 0.5% by weight to provide improved performance.

In some configurations, the surfactant can include at least one material selected from the group that includes polyethylene glycol ester condensates and alkyl glycoside surfactants. For example, the surfactant can be a GLUCOPON surfactant, available from Cognis Corporation (having a place of business in Cincinnati, Ohio, U.S.A.) which can be composed of 40% by weight water, and 60% by weight d-glucose, decyl, octyl ethers and oligomerics.

The absorbent article 20 may also include other components, such as fluid wicking layers, intake layers, surge layers, distribution layers, transfer layers, barrier layers, wrapping layers and the like, as well as combinations thereof. For example, with reference to FIG. 5, a surge layer 32 in one aspect may be positioned between the topsheet 26 and the absorbent layer 30.

In other aspects of the invention, the surfactant can be in the form of a sprayed-on surfactant comprising a water/surfactant solution which includes 16 liters of hot water (about 45° C. to 50° C.) mixed with 0.20 kg of GLUCOPON 220 UP surfactant available from Cognis Corporation and 0.36 kg of AHCHOVEL Base N-62 surfactant available from Uniqema (having a place of business in New Castle, Del., U.S.A.).

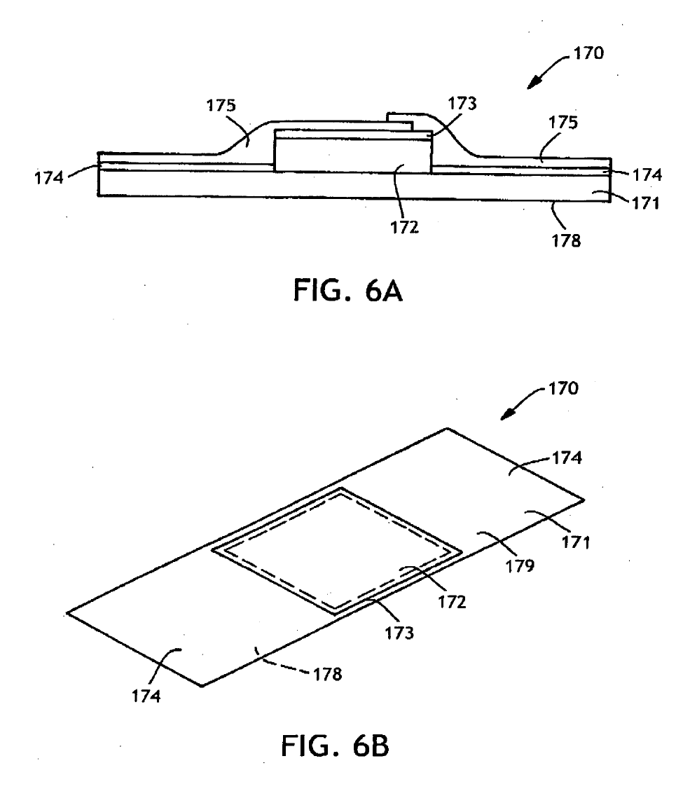

In addition to the absorbent articles described above, the absorbent articles of the present invention may be used as an absorbent bandage. Attention is directed to FIGS. 6A and 6B, which show a possible configuration for a bandage of the present invention. FIG. 6A shows a cross-section view of the absorbent bandage with optional layers described below. FIG. 6B shows a perspective view of the bandage of the present invention with some of the optional or removable layers not being shown. The absorbent bandage 170 has a strip 171 of material having a body-facing side 179 and a second side 178 which is opposite the body-facing side. The strip is essentially a backsheet and is desirably prepared from the same materials described above for the backsheet. In addition, the strip may be an apertured material, such as an apertured film, or material which is otherwise gas permeable, such as a gas permeable film. The strip 171 supports the absorbent layer 172 of the present invention which is attached to the body facing side 179 of the strip. In addition, an optional absorbent protective layer 173 may be applied to the absorbent layer 172 and can be coextensive with the strip 171. The absorbent layer 172 contains the fissured superabsorbent polymer composition of the present invention.

The absorbent bandage 170 of the present invention may also have a pressure sensitive adhesive 174 applied to the body-facing side 179 of the strip 171. Any pressure sensitive adhesive may be used, provided that the pressure sensitive adhesive does not irritate the skin of the user. Suitably, the pressure sensitive adhesive is a conventional pressure sensitive adhesive which is currently used on similar conventional bandages. This pressure sensitive adhesive is preferably not placed on the absorbent layer 172 or on the absorbent protective layer 173 in the area of the absorbent layer 172. If the absorbent protective layer is coextensive with the strip 171, then the adhesive may be applied to areas of the absorbent protective layer 173 where the absorbent layer 172 is not located. By having the pressure sensitive adhesive on the strip 171, the bandage is allowed to be secured to the skin of a user in need of the bandage. To protect the pressure sensitive adhesive and the absorbent, a release strip 175 can be placed on the body facing side 179 of the bandage. The release liner 175 may be placed on the body facing side of the bandage in a single piece (not shown) or in multiple pieces, as is shown in FIG. 6A.

In another aspect of the present invention, the absorbent layer of the bandage may be placed between a folded strip. If this method is used to form the bandage, the strip is suitably fluid permeable.

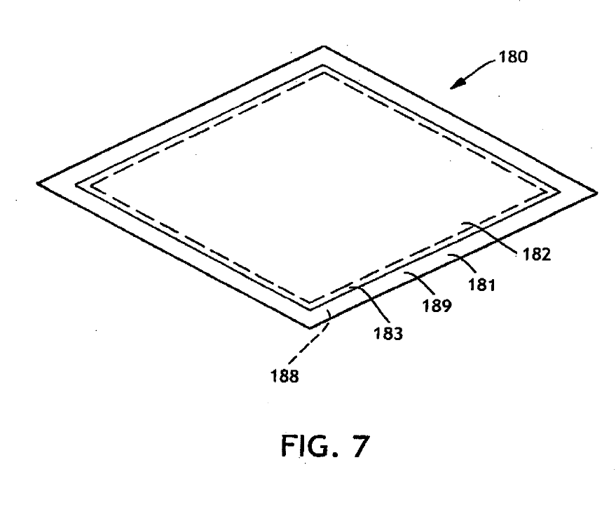

Absorbent furniture and/or bed pads or liners are also included within the present invention. As is shown in FIG. 7, a furniture or bed pad or liner 180 (hereinafter referred to as a “pad”) is shown in perspective. The pad 180 has a liquid impermeable backsheet 181 having a furniture-facing side or surface 188 and an upward facing side or surface 189 which is opposite the furniture-facing side or surface 188. The liquid impermeable backsheet 181 supports the absorbent layer 182 of the present invention which is attached to the upward facing side 189 of the liquid impermeable backsheet. In addition, an optional absorbent protective layer 183 may be applied to the absorbent layer. The absorbent layer contains the fissured superabsorbent polymer composition of the present invention. The substrate of the absorbent layer can be the liquid impermeable layer 181 or the absorbent protective layer 183 of the pad. In the alternative, in aspects where the absorbent layer has additional substrate, the layers of the absorbent layer can include the liquid impermeable layer 81, the superabsorbent polymer composition layer 182 and the absorbent protective layer 83.

To hold the pad in place, the furniture-facing side 188 of the pad may contain a pressure sensitive adhesive, a high friction coating or other suitable material which will aid in keeping the pad in place during use. The pad of the present invention can be used in a wide variety of applications including placement on chairs, sofas, beds, car seats and the like to absorb any fluid which may come into contact with the pad.

The present invention may be better understood with reference to the following examples.

Test Procedures

Centrifuge Retention Capacity (CRC) Test

As used herein, the Centrifuge Retention Capacity (CRC) is a measure of the Absorbent Capacity of the superabsorbent polymer composition retained after being subjected to centrifugation under controlled conditions. The CRC can be measured by placing a sample of the material to be tested into a water-permeable bag that will contain the sample while allowing the test solution (0.9 percent NaCl solution) to be freely absorbed by the sample. A heat-sealable tea bag material (available from Dexter Nonwovens of Windsor Locks, Conn., U.S.A., as item #11697) works well for most applications. The bag is formed by folding a 5-inch by 3-inch (12.7 cm×7.6 cm) sample of the bag material in half and heat sealing two of the open edges to form a 2.5-inch by 3-inch (6.4 cm×7.6 cm) rectangular pouch. The heat seals should be about 0.25 inch (0.64 cm) inside the edge of the material. After the sample is placed in the pouch, the remaining open edge of the pouch is also heat-sealed. Empty bags are also made to be tested with the sample bags as controls. A sample size is chosen such that the teabag does not restrict the swelling of the material, generally with dimensions smaller than the sealed bag area (about 2-inch by 2.5-inch) (5.1 cm×6.4 cm). Three sample bags are tested for each material.

The sealed bags are submerged in a pan of 0.9 percent by weight NaCl solution. After wetting, the samples remain in the solution for 60 minutes, at which time they are removed from the solution and temporarily laid on a non-absorbent flat surface.

The wet bags are then placed into the basket of a suitable centrifuge capable of subjecting the samples to a g-force of 350. (A suitable centrifuge is a HERAEUS LABOFUGE 400, Heraeus Instruments, part number 75008157, available from Heraeus Infosystems GmbH, Hanau, Germany). The bags are centrifuged at a target of 1600 rpm, but within the range of 1500-1900 rpm, for 3 minutes (target g-force of 350). The bags are removed and weighed. The amount of fluid absorbed and retained by the material, taking into account the fluid retained by the bag material alone, is the Centrifuge Retention Capacity of the material, expressed as grams of fluid per gram of material.

Composite Centrifuge Retention Capacity (CCRC) Test

The Composite Centrifuge Retention Capacity Test is conducted in the same manner as the Centrifuge Retention Capacity Test above, with one exception. Rather than placing a sample of the superabsorbent material to be tested into a water-permeable bag, a 4.4 cm diameter circle of substrate coated with the superabsorbent material is placed into the water-permeable bag. All other steps in the procedure remain the same. The amount of fluid absorbed and retained by the coated substrate, taking into account the fluid retained by the bag material alone, is the Composite Centrifuge Retention Capacity of the material, expressed as grams of fluid per square meter of material.

Composite Centrifuge Retention Capacity (CCRC) Index Test

The Composite Centrifuge Retention Capacity Index Test is conducted in the same manner as the Composite Centrifuge Retention Capacity Test above. However, the results are normalized to account for the basis weight of the absorbent layer. Therefore, the CCRC results expressed in g/m² are then divided by the basis weight of the absorbent layer expressed in g/m². The result is the CCRC Index.

Plate Stiffness Test

Stiffness of the absorbent layer composites were measured using the “Zwick Flexibility” test. This test is a measure of stiffness of an article as it is deformed downward into a hole beneath the sample. For the test, the sample is modeled as an infinite plate with thickness t that resides on a flat surface where it is centered over a hole with radius R. A central force applied to the foam directly over the center of the hole deflects the foam down into the hole by a distance w when loaded in the center by a Force F. For a linear elastic material the deflection can be predicted by:

$$w = \frac{3 F}{4 π {Et}^{3}} ( 1 - v ) ( 3 + v ) {R}^{2}$$

where E is the effective linear elastic modulus, v is the Poisson's ratio, R is the radius of the hole, and t is the thickness of the foam, taken as the caliper in millimeters measured under a load of about 0.35 kPa, applied by a 7.6 cm diameter Plexiglass platen, with the thickness measured with a Sony U60A Digital Indicator. Taking Poisson's ratio as 0.1 (the solution is not highly sensitive to this parameter, so the inaccuracy due to the assumed value is likely to be minor), the previous equation can be rewritten for w to estimate the effective modulus as a function of the flexibility test results:

$$E \approx \frac{2 {R}^{2}}{3 {t}^{3}} \frac{F}{w}$$

The test results are carried out using an MTS Alliance RT/1 testing machine (MTS Systems Corp., having a place of business in Eden Prairie, Minn., U.S.A.) with a 100 N load cell. As an absorbent composite at least 6.25 cm by 6.25 cm square sits centered over a hole of radius 17 mm on a support plate, a blunt probe of 3.15 mm radius descends at a speed of 2.54 mm/min. When the probe tip descends to 1 mm below the plane of the support plate, the test is terminated. The maximum slope in grams of force/mm over any 0.5 mm span during the test is recorded (this maximum slope generally occurs at the end of the stroke). The load cell monitors the applied force and the position of the probe tip relative to the plane of the support plate is also monitored. The peak load is recorded, and E is estimated using the above equation.

The bending stiffness per unit width can then be calculated as:

$$S = \frac{{Et}^{3}}{12}$$

### EXAMPLES

### Example 1

### Superabsorbent Polymer Solution

The superabsorbent polymer composition was prepared as described below.

Two monomer solutions were prepared separately. Solution No. 1 was prepared as follows: to 237 grams (3.289 moles) of acrylic acid was added 31.5 grams polyethylene glycol (molecular weight=200) and 52.6 grams of sodium hydroxide in 350 grams of water (40% neutralization) and 1.5 grams of ascorbic acid. This solution was then mixed and cooled in an ice bath.

Solution No. 2 was prepared as follows: 31.5 grams polyethylene glycol (mol. wt.=200) was diluted with 200 g water, then, with rapid stirring, 5 ml of 3-(trimethoxysilyl)propyl methacrylate(2.7×10-2 moles) was added to produce a hazy solution. To this solution was added 3.15 g of 30% aqueous hydrogen peroxide. This solution was then mixed.

A third solution was prepared by dissolving 39.5 grams (0.987 moles) sodium hydroxide in 300 grams of water. This solution was then mixed.

Solution No. 2 was added to Solution No. 1 in an ice bath while stirring with a magnetic stir bar. A thermocouple was used to monitor the temperature and observe the reaction exotherm. The polymerization reaction began after about 5 minutes of mixing. Once the exotherm reaction was detected, water was added gradually to keep the solution viscosity suitable for stirring. A total of 450 grams of water was added over 20 minutes. A maximum polymerization temperature of 85° C. was observed about 8 minutes after mixing of the two monomer solutions. After about 20 minutes, solution 3 was added with stirring to bring the neutralization to 70%, and was followed by additional water to reduce the polymer concentration to about 20% by weight.

### Example 2

### Absorbent Layer

A larger batch of the superabsorbent polymer solution was prepared using the same proportions and the same steps as in Example 1; however, the amount of water in the last step was adjusted to form a 29.8% by weight polymer solution. The superabsorbent polymer composition solution was placed into a drum of a pilot machine and fed to a continuous slot die having a slot width of 41 inches (104 cm) wide with a positive displacement pump. The 29.8% solution of the superabsorbent polymer composition was applied at about 120 gsm to a 58 inch (147 cm) wide substrate moving at 100 feet/min (30.5 m/min). The substrate was a 20 gsm polypropylene surge nonwoven with side by side polyethylene/polypropylene (PE/PP) bicomponent fibers (available from Kimberly-Clark Corporation, having a place of business in Neenah, Wis., U.S.A.). A suitable method for making this substrate is described in U.S. Publication No. US 2005/0098256 to Polanco et al., which was previously incorporated by reference in a manner that is consistent herewith. The coated substrate was then dried in a two-zone 40-foot (12.2 m) flotation drier to provide a necked absorbent layer having a dry coating of 35 gsm. The air temperature in the first zone was 154° C., the air temperature in the second zone was 160° C., and the exit temperature of the absorbent layer was 110° C. During the drying process, the coated sections of the web narrowed from 41 inches (104 cm) to 30 inches (76 cm), which is equivalent to about 73% of the original web width in the cross-machine direction.

A 10 cm by 10 cm sample of this necked material was pulled back out (i.e., restretched) by hand to gain back the material that was lost in the necking process. This restretch procedure fissured the dry superabsorbent polymer composition, although it remained securely attached to the substrate. The absorbent layer was then measured for thickness and density, and was tested for stiffness and absorbent capacity, the results of which can be seen in the Table 1.

TABLE 1

| | Thickness | Density | Stiffness | CCRC |
|---|---|---|---|---|
| Sample | (mm) | (g/cm³) | (N · mm) | (g/m²) |
| Necked | .97 | .092 | 2.89 | 794 |
| Necked and | .83 | .092 | 0.37 | 1200 |
| restretched | | | | |

It can be seen that by restretching the necked absorbent layer, the thickness decreased by about 14%. The density remained constant. The stiffness decreased by about 87% as measured by the Plate Stiffness Test, and the absorbent capacity increased by about 51% as measured by the Composite Centrifuge Retention Capacity Test.

### Example 3

### Absorbent Layer with Additional Layer

A larger batch of the superabsorbent polymer solution was prepared using the same proportions and the same steps as in Example 1; however, the amount of water was adjusted in the last step to form a 34% by weight polymer solution. The superabsorbent polymer composition was placed into a drum of a pilot machine and fed to a continuous slot die having a slot width of 41 inch (104 cm) wide with a positive displacement pump. The 34% solution of superabsorbent polymer composition solution was applied at about 180 gsm to a 58 inch (147 cm) wide substrate moving at 100 feet/min (30.5 m/min). The substrate was an 18 gsm spunbond nonwoven (such as a diaper liner currently found in HUGGIES diapers, manufactured by Kimberly-Clark Corporation, having a place of business Neenah, Wis., U.S.A.). An additional layer of the 18 gsm spunbond nonwoven fabric was laid on top of the wet coating to form a spunbond/superabsorbent/spundbond sandwich. The resulting laminate was dried in a two-zone 40-foot (12.2 m) flotation drier to provide a necked absorbent layer having a dry coating of 60 gsm. The air temperature in the first zone was 154° C., the air temperature in the second zone was 160° C., and the exit temperature of the absorbent layer was 110° C. During the drying process, the coated sections of the web narrowed from 41 inches (104 cm) to 27 inches (69 cm) or 65% of the original web width.

A 30 cm by 50 cm sample of this necked material was passed between grooved rolls similar to those described in U.S. Pat. No. 4,285,100 to Schwarz (previously incorporated by reference in a manner that is consistent herewith) to gain back the CD length that was lost in the necking process. The extent of roll engagement (i.e., the distance that the rolls overlap) was 3.3 mm. This restretch procedure increased the CD width by 35% and fissured the dry superabsorbent polymer composition, although it remained securely attached within the substrates. The absorbent layer was then measured for thickness and density, and was tested for stiffness and absorbent capacity, the results of which can be seen in the Table 2.

TABLE 2

| | Thickness | Density | Stiffness | CCRC | CCRC |
|---|---|---|---|---|---|
| Sample | (mm) | (g/cm³) | (N · mm) | (g/m²) | Index |
| Necked | .52 | .256 | 5.0 | 886 | 6.7 |
| Necked and | .50 | .207 | 0.4 | 782 | 7.6 |
| restretched | | | | | |

It can be seen that by restretching the necked absorbent layer, the thickness decreased by about 3%. The density decreased by about 19%. The stiffness decreased by about 92% as measured by the Plate Stiffness Test, and the absorbent capacity index increased by about 13% as measured by the Composite Centrifuge Retention Capacity Index Test.

It will be appreciated that details of the foregoing examples, given for purposes of illustration, are not to be construed as limiting the scope of this invention. Although only a few exemplary embodiments of this invention have been described in detail above, those skilled in the art will readily appreciate that many modifications are possible in the examples without materially departing from the novel teachings and advantages of this invention. For example, features described in relation to one example may be incorporated into any other example of the invention.

Accordingly, all such modifications are intended to be included within the scope of this invention, which is defined in the following claims and all equivalents thereto. Further, it is recognized that many embodiments may be conceived that do not achieve all of the advantages of some embodiments, particularly of the preferred embodiments, yet the absence of a particular advantage shall not be construed to necessarily mean that such an embodiment is outside the scope of the present invention. As various changes could be made in the above constructions without departing from the scope of the invention, it is intended that all matter contained in the above description shall be interpreted as illustrative and not in a limiting sense.

What is claimed is:

1. An absorbent article comprising:
a backsheet; and
an absorbent layer adjacent to and in facing relationship with the backsheet;
wherein the absorbent layer comprises a support substrate and a superabsorbent polymer composition;
wherein the superabsorbent polymer composition comprises a water-soluble ionic polymer capable of sufficient non-radiative crosslinking within about 10 minutes at a temperature of about 120° C. or less to reach an absorbent capacity of at least 1 g/g as measured by the Centrifuge Retention Capacity Test; and
wherein the absorbent layer has been restretched.

2. The absorbent article of claim 1 wherein the superabsorbent polymer composition comprises:
about 15 to about 99.9% by mass monoethylenically unsaturated polymer units;
about 0.1 to about 20% by mass ester units selected from the group consisting of acrylate and methacrylate ester units that include an alkoxysilane functionality; and
zero to about 75% by mass of units selected from the group consisting of polyolefin glycol and polyolefin oxide units.

3. The absorbent article of claim 1 wherein the superabsorbent polymer composition is substantially fissured.

4. The absorbent article of claim 1 wherein the absorbent layer has a density of about 0.1 g/cc or less.

5. The absorbent article of claim 1 wherein the absorbent layer exhibits a decrease in stiffness as measured by the Plate Stiffness Test of at least about 50% when compared to a similar unrestretched absorbent layer.

6. The absorbent article of claim 1 wherein the absorbent layer has a stiffness of about 2 N·mm or less as measured by the Plate Stiffness Test.

7. The absorbent article of claim 1 wherein the absorbent layer exhibits an increase in absorbent capacity as measured by the Composite Centrifuge Retention Capacity Test of at least about 30% when compared to a similar unrestretched absorbent layer.

8. The absorbent article of claim 1 wherein the absorbent layer has an absorbent capacity of at least about 800 g/m² as measured by the Composite Centrifuge Retention Capacity Test.

9. The absorbent article of claim 1 wherein the absorbent layer has a thickness of about 1 mm or less.

10. The absorbent article of claim 1 wherein the absorbent layer has a CD wet strength of at least about 11 N as measured by ASTM D-5034.

11. The absorbent article of claim 1 further comprising a topsheet, wherein the topsheet is positioned such that the absorbent layer is positioned between the topsheet and the backsheet.

12. The absorbent article of claim 11 wherein at least one of the backsheet, the absorbent layer and the topsheet is stretchable.

13. The absorbent article of claim 1 wherein the support substrate is selected from films, spunbond nonwoven webs, surge materials, bonded-carded-webs, bonded-airlaid webs, coform, woven fabrics, or laminates thereof.

14. The absorbent article of claim 1 wherein the absorbent layer further comprises an additional substrate positioned such that the superabsorbent polymer composition is positioned between the support substrate and the additional substrate.

15. The absorbent article of claim 14 wherein the additional substrate is selected from films, spunbond nonwoven webs, surge materials, bonded-carded-webs, bonded-airlaid webs, coform, woven fabrics, or laminates thereof.

16. The absorbent article of claim 14 wherein the absorbent layer has a density of about 0.3 g/cc or less.

17. The absorbent article of claim 14 wherein the absorbent layer exhibits a decrease in stiffness as measured by the Plate Stiffness Test of at least about 50% when compared to a similar unrestretched absorbent layer.

18. The absorbent article of claim 14 wherein the absorbent layer has a stiffness of about 2 N·mm or less as measured by the Plate Stiffness Test.

19. The absorbent article of claim 14 wherein the absorbent layer exhibits an increase in absorbent capacity index as measured by the Composite Centrifuge Retention Capacity Index Test of at least about 10% when compared to a similar unrestretched absorbent layer.

20. The absorbent article of claim 14 wherein the absorbent layer has an absorbent capacity index of at least about 7 as measured by the Composite Centrifuge Retention Capacity Index Test.

21. The absorbent article of claim 14 wherein the absorbent layer has a thickness of about 2 mm or less.

22. The absorbent article of claim 14 wherein the absorbent layer has a CD wet strength of at least about 11 N as measured by ASTM D-5034.

23. The absorbent article of claim 1 wherein the absorbent article is selected from personal care absorbent articles, health/medical absorbent articles or household/industrial absorbent articles.
